(19) United States
(12) Patent Application Publication
(10) Pub. No.: US 2025/0041334 A1
(43) Pub. Date: Feb. 6, 2025

(54) Compositions for Virulence Suppression

(71) Applicant: Solventum Intellectual Properties Company, Maplewood, MN (US)

(72) Inventors: Ranjani V. Parthasarathy, Woodbury, MN (US); Max A. Kruziki, New Brighton, MN (US); Hannah C. Cohen, St. Paul, MN (US)

(21) Appl. No.: 18/718,158

(22) PCT Filed: Nov. 28, 2022
(86) PCT No.: PCT/IB2022/061498

§ 371 Date: Jun. 10, 2024

Related U.S. Application Data
(60) Provisional application No. 63/265,203, filed on Dec. 10, 2021.

(51) Int. Cl. A61K 33/32; A61K 33/26; A61K 33/30; A61K 47/02; A61K 47/34; A61P 31/04

(52) U.S. Cl. CPC A61K 33/32; A61K 33/26; A61K 33/30; A61K 47/02; A61K 47/34; A61P 31/04

(57) ABSTRACT

A method of suppressing microbial virulence. The method includes administering and/or applying a composition. The composition includes (a) a surfactant comprising (i) a polyethylene glycol compound comprising a fatty acid moiety, or (ii) a fatty acid containing compound. The composition further includes (b) (i) a phosphate containing compound, and (ii) a metal salt; and (c) an aqueous carrier. The surfactant is present in an amount of between 0.01 wt. % and 1 wt. %, based on the total weight of the composition.

### BACKGROUND

Compositions for virulence suppression are described in, for example, U.S. Patent Application Publication No. 2019/0247423 to Alverdy et al.

### DETAILED DESCRIPTION

Many known treatments of pathogens result in the destruction of all or nearly all microbes that may be present, even beneficial microbes. Further, because of these treatment methods, there is growing concern about antibiotic resistance that may increase risks to patients, particularly to those undergoing surgical procedures. Newer approaches have been directed toward suppressing the virulence of the pathogen that causes the infection rather than destroying all microbes. That is, new methods are needed that do not destroy all the beneficial bacteria in the process of preventing the harm done by pathogens.

Antiseptics like chlorhexidine gluconate (CHG) are used typically to bathe the patient prior to surgery as well as in the ICU. There is a growing recognition that there is an overuse of CHG in the ICU that can result in issues such as contact dermatitis as well as in some cases increased antimicrobial resistance. It may be useful to have a product that can remove microbes and leave behind a solution that can suppress the virulence of the remaining microbes on skin.

In another application, saline or dilute betadine is used to wash a wound or surgical cavity. Rather than disturb the microbiome, it may be useful to have products that are useful for suppressing the virulence of microbes in the wound/surgical cavity. This concept can be extended to other microbiomes—vaginal, gut, nasal/oral microbiomes.

### Definitions

The term “metal salt” refers to all metal salts except metal salts that include sodium (e.g., NaCl).

The term “μM” used herein refers to micromolar concentration.

The term “mM” used herein refers to millimolar concentration.

The term “virulence” refers to a pathogen's ability to infect or damage a host such as a mammal.

The term “virulence suppression” and “suppression of microbial virulence” or similar expressions refer to suppressing or inhibiting the synthesis and/or expression of one or more virulence factors.

The term “virulence factor” refers to molecules produced by microbes that enable them to infect a host such as a mammal. The virulence factors of bacteria can be small molecules, proteins, or biofilms (e.g., a slimy buildup of bacteria on a surface). The virulence factors are typically secreted by a microbe to promote colonization and/or adhesion to a host (e.g., resulting in biofilm formation), to evade the immune response of the host, or to obtain nutrients from the host.

As used herein, “surfactant” refers to synthetic and naturally occurring amphiphilic molecules that have hydrophobic portion(s) and hydrophilic portion(s). Due to their amphiphilic (amphipathic) nature, surfactants typically can reduce the surface tension between two immiscible liquids, for example, the oil and water phases in an emulsion, stabilizing the emulsion. Surfactants can be characterized based on their relative hydrophobicity and/or hydrophilicity. For example, relatively lipophilic surfactants are more soluble in fats, oils and waxes, and typically have HLB (hydrophile-lipophile balance) values less than or about 10, while relatively hydrophilic surfactants are more soluble in aqueous compositions, for example, water, and typically have HLB values greater than or about 10. Relatively amphiphilic surfactants are soluble in oil- and water-based liquids and typically have HLB values close to 10.

The terms “comprise”, “contain”, “include”, and variations thereof do not have a limiting meaning where these terms appear in the description and claims. Such terms will be understood to imply the inclusion of a stated step or element or group of steps or elements but not the exclusion of any other step or element or group of steps or elements. By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase and is limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether they materially affect the activity or action of the listed elements. Any one of the elements or combinations of elements that are recited in this specification in open-ended language (e.g., comprise, include, contain, and derivatives thereof), are considered to additionally be recited in closed-ended language (e.g., consist and derivatives thereof) and in partially closed-ended language (e.g., consist essentially, and derivatives thereof).

In this application, terms such as “a”, “an”, and “the” are not intended to refer to only a singular entity but include the general class of which a specific example may be used for illustration. The terms “a,” “an,” and “the” are used interchangeably with the term “at least one.” The phrases “at least one of” and “comprises at least one of” followed by a list refers to any one of the items in the list and any combination of two or more items in the list.

As used herein, the term “or” is generally employed in its usual sense including “and/or” unless the content clearly dictates otherwise. The term “and/or” means one or both. For example, the expression A and/or B means A alone, B alone, or both A and B.

Also, the recitations of numerical ranges by endpoints include all numbers subsumed within that range as well as the endpoints (e.g., 1 to 5 includes 1, 1.5, 2, 2.75, 3, 3.80, 4, 5, etc.) and any sub-ranges (e.g., 1 to 5 includes 1 to 4, 1 to 3, 2 to 4, etc.).

In some embodiments, the present disclosure is directed to compositions for suppressing microbial virulence. The virulence suppression compositions can suppress the expression of various virulence factors without destroying all microbes that may be present.

In some embodiments, microbial virulence may be suppressed by reducing or inhibiting the formation and/or expression of one or more virulence factors, which are the harmful products that can lead to microbial infections. That is, the virulence suppression compositions can prevent, mitigate, or treat microbial infections. The compositions typically do not prevent continued colonization of microbes such as those that are helpful to the mammal.

In some embodiments, the virulence suppression compositions may include (i) a surfactant comprising (a) a polyethylene glycol compound comprising a fatty acid moiety, and/or (b) fatty acid containing compound; (ii) (a) phosphate containing compound and/or (b) a metal salt; and (iii) an aqueous carrier. Surprisingly, it was discovered that at even very low levels of the above-described surfactants, when used in combination with either or both of a phosphate or metal salt, the compositions of the present disclosure may suppress certain virulence factors that lead to microbial infections.

Generally, the microbial virulence suppression compositions of the present disclosure may include polyethylene glycol (PEG) compounds that contain fatty acids esters/ethers/amides/monoalkyl glycols or fatty acid esters/ethers/monoalkyl glycols or combinations thereof that are physiologically well tolerated after administration to skin/mucosa/wound/surgical cavity/peritoneal cavity or gut. These components may be selected to be therapeutically acceptable, which means that they are not toxic to the mammal being treated.

In some embodiments, the microbial virulence suppression compositions of the present disclosure may include a surfactant that includes one or more polyethylene glycol (PEG) compounds. PEG compounds, also referred to herein as PEGs, together with their derivatives, do not have definite chemical entities, but are compound mixtures having different chain lengths. PEG includes two terminal primary hydroxyl groups that can be used to create mono-, di- and poly-esters, amines, ethers and acetals. PEGs can also create additional compounds and complexes through a reaction in their ether bridges.

In the present application, the term PEG compound refers to PEGs and PEG derivatives such as, for example, PEG alkyl ethers (e.g., laureths, ceteths, ceteareths, oleths, and PEG ethers of glyceryl cocoates), PEG alkyl esters (e.g., PEG laurates, dilaurates, stearates, and distearates), PEG castor oils, PEG alkyl amides (e.g., PEG cocamines), PEG propylene glycols, PEG 1,2 diols, and other derivates (e.g., PEG soy sterols and PEG beeswax). Since many PEG types are hydrophilic, they are effective penetration enhancers for use in dermatological preparations. The PEG compounds may be used alone or in combination, or may be used with optional compounds such as any of alkyl esters, alkyl ethers, and alkyl amides, and mixtures and combinations thereof. Any of the alkyl esters, alkyl ethers and alkyl amides can have an alkyl group independently selected to have 8 to 22 carbon atoms. In some embodiments, the alkyl group can include a 1,2 dihydroxy group.

In some embodiments, the PEG compounds of the present disclosure may have surfactant properties, and the HLB value (Hydrophile-Lipophile Balance) is used in the present application as an empirical expression for the relationship of the hydrophilic and hydrophobic groups of the PEG compound (or any other surfactant in the composition), and in most cases the higher the HLB value, the more water-soluble the surfactant.

In some embodiments of the present disclosure, HLB values are calculated using the method of Griffin (Griffin W C; J. Soc. of Cosmetic Chemists, pp. 249-256 (1954)). Thus, as used herein, the “HLB Method” involves a calculation based on the following: HLB=(E+P)/5, where E is the weight percent of oxyethylene content and P is the weight percent of alcohol content (glycerol, sorbitol, etc.). For the compounds herein, glycerol segments with two hydroxyl groups, glycerol segments with one hydroxyl group, and hydroxyl-containing segments of any additional polyhydric molecules were included in the definition of P.

Other methods of calculating the HLB value are available and may be required when determining the HLB value for compounds lacking both E and P groups, as defined above. While the calculated value of HLB may vary depending on the method used, the trends and relative hydrophobicity of materials are expected to be similar.

In various embodiments, the PEG compounds of the present may have an HLB value of greater than 8 and less than 18, or greater than 8 and less than 14, or greater than 10 and less than 14.

In various embodiments, the PEG compound may include PEG alkyl esters, PEG alkyl ethers, PEG alkyl amides, PEG 1,2 diols, and mixtures and combinations thereof, wherein where the alkyl group on any of the PEG compounds can be independently selected to have 8 to 22 carbon atoms. In some embodiments, the alkyl groups on any of the PEG compounds can include a 1,2 dihydroxy group.

In some embodiments, the PEG compound may include a PEG alkyl ester with an alkyl group having 8 to 22 carbon atoms. The PEG alkyl esters, which can also be referred to in the art as PEG fatty acid esters, are the reaction products of a PEG compound (hereafter referred to as a PEG) and a fatty acid. The PEG in the PEG alkyl ester forms a hydrophilic part of the molecule and the C8-C22 alkyl ester component of the PEG alkyl ester forms a lipophilic part of the molecule. By varying the molecular weight of the PEG and the alkyl ester components of the PEG alkyl ester, surfactants covering a wide range of HLB values can be produced. In various embodiments, the PEG alkyl ester is a monoester, a diester or a triester, or a mixture or combination thereof. In some embodiments, the PEG alkyl ester is substantially free of triesters, and in some embodiments the PEG alkyl ester is substantially free of both triesters and diesters, and as such consists substantially of monoesters.

In various embodiments, the PEG compound may include a PEG alkyl ester that is a reaction product of a fatty acid component chosen from C8 to C22 fatty acids (fatty acids with 8 to 22 carbon atoms), or C8 to C18 fatty acids, or C8 to C12 fatty acids; and a PEG component with 6 to 60 ethylene oxide units, or 6 to 40 ethylene oxide units, or 6 to 32 ethylene oxide units, or 6 to 10 ethylene oxide units.

In some embodiments, the PEG alkyl ester may be a reaction product of an ethoxylated glyceride and a fatty acid. In some embodiments, the fatty acid may be a C8 to C22 fatty acid, and a PEG with 6 to 60 ethylene oxide units. In another embodiment, the PEG alkyl ester is a reaction product of a C8 to C18 fatty acid, and a PEG with 6 to 32 ethylene oxide units.

In various example embodiments, the PEG alkyl ester may be chosen from PEG 12 glyceryl laurate, PEG 20 glyceryl laurate, PEG 30 glyceryl laurate, PEG 20 glyceryl stearate, PEG glyceryl caprate, and mixtures and combinations thereof. In any of the embodiments above, the PEG alkyl ester can include a mixture of mono, di, and tri esters. In some embodiments, the PEG alkyl ester includes a mixture of monoesters and diesters, and is substantially free of tri esters, or free of tri esters. In some embodiments, the PEG alkyl ester includes monoesters and is substantially free of diesters and triesters, or free of diesters and triesters.

In some example embodiments, the PEG alkyl ester is a reaction product of a PEG and a C8 to C22 fatty acid triester, a reaction product of a PEG and a C8 to C22 fatty acid monoester or diester, or a reaction product of a PEG, a C8 to C22 fatty acid monoester or diester, and a fatty acid mono, di and triester.

Suitable commercially available PEG glyceryl alkyl esters include, but are not limited to, those available under the trade designation LABRAFIL from Gattefossé, Lyon, FR, (HLB=9), LABRASOL from Gattefossé (HLB=12), and TEFOSE from Gattefossd (HLB=9-10) and peg glyceryl laurate from Global 7

Suitable commercially available peg fatty acid esters include peg 12 laurate and MYRJ from Gattefosse.

In various embodiments, PEG compounds may be present in the virulence suppression compositions in an amount of between 0.01 wt % and 10 wt %, between 0.01 wt. % and 2 wt %, or between 0.01 wt % and 1 wt %, or between 0.01 wt % and 0.1 wt %, based on the total weight of the virulence suppression composition. In some, embodiments, PEG compounds may be present in the composition in an amount such that the presence of the PEG compounds does not appreciably contribute to an antimicrobial characteristic of the compositions (i.e., the PEG compounds are provided at low enough concentrations such that, in the absence of other antimicrobial components in the compositions, the compositions may allow for microbial growth). In some cases, they may have delayed growth of organisms.

In some embodiments, the virulence suppression compositions of the present disclosure may include (in addition to or in lieu of PEG compounds) a surfactant that includes one or more fatty acid containing compounds (as used herein, unless expressly associated with the term “PEG”, the term “fatty acid containing compounds” does not include PEG compounds containing fatty acid moieties). In some embodiments, suitable fatty acid containing compounds can include esters, ethers, amides, or monoalkyl glycols with the number of carbons in the alkyl group from 8 to 22.

In some embodiments, suitable fatty acid esters may be monoesters, diesters, triesters, or a mixture or combination thereof. In some embodiments, the fatty acid ester may be chosen from monoester and diesters, and mixtures and combinations thereof. In some embodiments, the fatty acid ester is a monoester. In some embodiments, suitable fatty acid esters may include those available under the trade designation CAPMUL from Abitec, Columbus, OH (HLB=6), geleol, Precirol ATO5, Maisine cc, Compritol 888 ATO, Sedefos 75 from Gattefosse.

In some embodiments, suitable fatty acid containing compounds include monoalkyl glycol (1,2 diols) and monoalkyl glycerol, or monoacyl glycerol where the number of carbons in the alkyl group can go from 8 to 22.

In some embodiments, the fatty acid containing compounds of the present may have an HLB value of greater than 8 and less than 18, or greater than 8 and less than 14, or greater than 10 and less than 14.

In various embodiments, fatty acid containing compounds may be present in the virulence suppression compositions in an amount of between 0.01 wt % and 10 wt %, between 0.01 wt. % and 2 wt %, or between 0.01 wt % and 1 wt %, or between 0.01 wt % and 0.1 wt %, based on the total weight of the virulence suppression composition. In some, embodiments, fatty acid containing compounds may be present in the composition in an amount such that the composition does not exhibit antimicrobial properties.

In some embodiments, the microbial virulence suppression compositions of the present disclosure may include either or both of one or more metal salts and one or more phosphate containing compounds. Generally, such components are believed to provide nutrients to the bacteria to reduce virulence, interact with other components of the composition through electrostatics to affect composition properties, and contribute to wound healing and reduction of infections by activating a host response (e.g., causing the body of a human host to create a response that may kill certain bacteria).

In some embodiments, suitable metal salts may include iron salts, manganese salts, zinc salts, rubidium salts, calcium salts, or a combination thereof. In some embodiments, the metals salts may include an iron salt. For example, the metal salts may include a mixture of metal salts that include iron salts, manganese salts, and zinc salts.

In some embodiments, metal salts may be present in the virulence suppression compositions in an amount of between 1 μM and 100 μM, between 5 μM and 80 μM, or between 10 μM and 40 μM, based on the total volume of the virulence suppression composition.

In some embodiments, suitable phosphates may include salts of sodium and potassium, and ammonium phosphate. In some embodiments, phosphates may be present in the virulence suppression compositions in an amount of between 1 mM and 100 mM, between 5 mM and 50 mM, or between 5 mM and 25 mM, based on the total volume of the virulence suppression composition.

In some embodiments, the incorporation of thickeners in the composition may delay the release of virulence suppression agents and therefore result in higher concentrations of those agents in the formulation. In this regard, in embodiments in which a thickener is present, metal salts may be present in the virulence suppression compositions in an amount of between 1 μM and 500 μM, between 1 μM and 200 μM, or between 5 μM and 200 μM based on the total volume of the virulence suppression composition. In embodiments in which a thickener is present, phosphates may be present in the virulence suppression compositions in an amount of between 1 mM and 1000 mM, between 5 mM and 700 mM, or between 5 mM and 500 mM, based on the total volume of the virulence suppression composition.

In various embodiments, the aqueous carrier may be present in the virulence suppression composition in an amount of between 5 wt % and 99 wt %, between 10 wt % and 98 wt %, between 10 wt % and 95 wt %, or between 10 wt % and 90 wt %, based on the total weight of the composition. In various embodiments, the aqueous carrier may include at least 80 wt % water, at least 90 wt % water, or at least 95 wt % water, based on the total weight of the aqueous carrier. In some embodiments, the aqueous carrier consists of water, which in this application means that the aqueous carrier is substantially 100 wt % water, or 100 wt % water, based on the total weight of the aqueous carrier.

In some embodiments, the aqueous carrier may include an alcohol chosen from benzyl alcohol, phenoxy ethanol, isopropyl alcohol, ethanol, and mixtures and combinations thereof. In various embodiments, alcohol may be present in the virulence suppression composition in an amount of between 0.005 wt. % and 10 wt. %, based on the total weight of the composition.

In some embodiments, the aqueous carrier in the antimicrobial composition may include a humectant. As used herein the term “humectant” refers to polar compounds or mixtures of compounds that act to retain or absorb moisture. Suitable humectants include, but are not limited to, polyols, such as glycerin, propylene glycol, dipropylene glycol, polypropylene glycol, glycerine ethoxylates, methyl glucose ethoxylates, polyethylene glycol, polyethylene/polypropylene glycols, and sorbitol. In some embodiments, the humectants include liquid polar solvents such as for example, monoalkyl glycols, glycerol alkyl ethers, monoacyl glycerols, and mixtures and mixtures and combinations thereof. Suitable examples of the liquid polar solvents include, but are not limited to, glycerol, propylene glycol, polyethylene glycol, pentylene glycol, and mixtures and combinations thereof.

In some embodiments, the aqueous carrier can be a mixture of water and a liquid glycol such as, for example, propylene glycol, pentylene glycol and mixtures thereof. For such mixtures, the ratio of water to glycol (or glycols) may be about 1:10 to about 10:1, or about 1:8 to about 8:1, or about 1:5 to about 5:1. Examples of useful aqueous carriers include water and pentylene glycol (2:1), water and propylene glycol (1:2) In various embodiments, liquid glycol may be present in the virulence suppression composition in an amount of between 1 wt % and 30 wt %, or between 1 wt % to about 20 wt %, based on the total weight of the composition.

In some embodiments, the addition of low levels of stabilizing ingredients in the aqueous carrier can also be advantageous. The addition of water-soluble gums such as guar derivatives, xanthan gum, and thickeners such as hydroxy ethyl cellulose, hydroxy propyl cellulose and carboxyl vinyl polymers may be helpful in stabilizing the virulence suppression composition. Suitable oil phase emulsion stabilizers include ethylene/acrylic acid copolymers such as those available under the trade designation AC540 from Allied Signal, Morrison, N.J., and N-vinyl pyrrolidone/olefin copolymers such as that available under the trade designation GANEX V-216 from ISP International Specialty Products, Wayne, N.J.

In some embodiments, the aqueous carrier may include predominantly aqueous solutions such as buffers.

In some embodiments, the virulence suppression compositions of the present disclosure (i) may not include any compounds that exhibit antimicrobial activity or (ii) may include compounds that can at certain concentrations exhibit antimicrobial activity, but are present at low enough concentrations such that they do not exhibit antimicrobial activity (i.e., the compositions may allow for microbial growth). For example, the compositions may not include any of the following: chlorhexidine salts; octenidine salts, benzalkonium chloride, parachlorometaxylenol (PCMX); triclosan; hexachlorophene; fatty acid monoesters of glycerin and propylene glycol such as glycerol monolaurate, glycerol monocaprylate, glycerol monocaprate, propylene glycol monolaurate, propylene glycol monocaprylate, propylene glycol monocaprate; phenols; surfactants and polymers that include a C12-C22 hydrophobe and a quaternary ammonium group; polyquatemary amines such as polyhexamethylene biguanide; quatemary silanes; hydrogen peroxide; silver and silver salts such as silver chloride, silver oxide and silver sulfadiazine iodine and its complexed forms, which are commonly referred to as iodophors. In some embodiments, the virulence suppression compositions can allow for microbial growth. In this regard, in some embodiments, when subjected to the Time-Kill test, the virulence suppression compositions may demonstrate a log reduction of microbes no greater than 3 logs, no greater than 2 logs, or no greater than 1 log. As used herein, the Time-Kill test is as carried out in accordance with the Time-Kill test of ASTM E2315.

In some embodiments, the virulence suppression compositions can also include components such as, for example, organic solvents, hydrophobic components (e.g., petrolatum and oils), hydrophilic components (glycerin and various ether and/or polyether compounds), silicones, carbohydrates (polysaccharides such as hydroxypropyl methyl cellulose), thickeners such as CARBOPOL, film-formers, emulsifiers, water, organic solvents (e.g., alcohols and polyols), stabilizers (e.g., polymers), fillers (e.g., organic materials such as polymeric particles and inorganic materials including ceramic particles, silica particles, clay particles, and glass particles), emollients/moisturizers, tonicity adjusting agents, chelating agents, anti-inflammatory agents, gelling agents, preservatives, pH adjusting agents, viscosity builders, time-release agents, dyes, fragrances or oils, and the like.

In some embodiments, thickeners, such as hydroxypropyl methylcellulose, can be employed to increase the viscosity of the compositions. In general, the polymers useful as thickeners have sufficient molecular weight to achieve thickening at generally less than 5 wt-% polymer, but not too high that the composition feels slimy and stringy. While the composition of the polymer will dramatically affect the molecular weight at which sufficient thickening will occur, the polymers may have a molecular weight of at least 250,000 daltons, or at least 500,000 daltons. The polymers may have a molecular weight of no greater than 3,000,000 daltons or no greater than 1,000,000 daltons.

Polymers used to thicken solutions can be classified as soluble, swellable, or associative in the aqueous compositions. Some polymers may fall into one or more of these classes. For example, certain associative polymers can be soluble in the aqueous system. Whether they are considered soluble, swellable, or associative in the aqueous system, suitable polymers may be film forming or not. Film forming polymers may retain the active virulence suppression component at the afflicted site for longer periods of time. This may be desirable for certain applications. For example, some film forming polymers may produce compositions that could not be easily washed off with water after being applied and dried.

As used herein, a soluble polymer is one that in dilute solution (i.e., 0.01-0.1 wt-% in the desired aqueous solvent system defined as containing water and any other hydrophilic compounds), after heating for a sufficient time to ensure solubilization of any potentially soluble components, has no significant observable particles of greater than 1 micron in particle size, as determined by light scattering measurements using, for example, Malvern Masterisizer E Laser Particle Size Analyzer available from Malvern Co., Boston, Mass.

As used herein, a swellable polymer is one that in dilute solution (i.e., 0.01-0.1 wt-% in the desired aqueous solvent system), after heating for a sufficient time to ensure solubilization of any potentially soluble components, has a significant (i.e., detectable) number of observable particles of greater than 1 micron in particle size, as determined by light scattering measurements using, for example, Malvem Masterisizer E Laser Particle Size Analyzer.

In some embodiments, the virulence suppression compositions may be suitable for treating any known microbe including, for example, bacteria, viruses, fungi such as Candida, and mycobacteria. In particular, administrating the medical composition can suppress virulence of at least one of gram negative Pseudomonas aeruginosa, gram positive Enterococcus faecalis, gram positive Staphylococcus aureus, or gram negative Serratia marcescens.

Unlike some previously known methods of treating microbial infections, the virulence suppression compositions of the present disclosure do not substantially kill all microbes within the treatment area. Although some of the pathogens may be destroyed at the treatment site such as those associated with a biofilm, colonization of the protective microbes is not substantially reduced. Stated differently, the pathogens can be contained and controlled while the colonization resistance of the non-pathogenic microbes and/or the normally protective microbes can be preserved. As used in reference to reduction in the number of microbes that are present, the term “substantially” means that there is less than 1 log reduction of the microbes. In some embodiments, there may be in increase in the growth of protective microbes.

In some embodiments of administering the virulence suppression composition, the virulence factor is reduced by at least 50 percent, at least 60 percent, at least 70 percent, at least 75 percent, at least 80 percent, at least 90 percent, at least 95 percent, at least 99 percent, at least 99.5 percent, or at least 99.9 percent when compared to the vehicle only control. The percentage can be based on weight, area, volume, or any other suitable measurable amount.

In some embodiments, the virulence suppression compositions can be administered and/or applied in various formulations such as a gel (e.g., cellulosic gel), a spray, lotion, ointment, solution, emulsion, dispersion, foam, coating, paste, powder, tablet, capsule, or the like. The formulation used can be chosen based upon the location of the infection or potential infection and on the desired delivery method.

In some embodiments, the virulence suppression compositions can be administered and/or applied in any desired formulation such as a spray, lotion, ointment, gel, solution, emulsion, dispersion, foam, coating, paste, powder, tablet, liquid, capsule, a drink or the like. The formulation used is dependent on the location of the infection or potential infection and on the desired delivery method.

For some applications, it is desirable that the virulence suppression compositions remain in a location where they are administered and/or applied. Such compositions are usually formulated to have a suitably high viscosity and/or to include a hydrophobic component that will enhance retention of the composition at the application location. These formulations can be, for example, an emulsion, ointment, gel, or lotion. Emulsions can be oil-in-water or water-in-oil.

By suppressing virulence, administration and/or application of the medical compositions can be used to prevent, mitigate, or treat a microbial infection.

In some embodiments, a method of suppressing microbial virulence is provided. The microbial virulence may be suppressed by reducing or inhibiting the synthesis and/or expression of one or more virulence factors by the microbe. By suppressing the synthesis and/or expression of one or more virulence factors, a microbial infection can be prevented, mitigated, or treated. The method includes administrating and/or applying any of the above-discussed virulence suppression compositions.

Any suitable method of administering and/or applying the virulence suppression compositions can be used. For example, the compositions can be applied to skin, mucosa, tissue (both exterior and interior surfaces of tissue), a wound site, a surgical site, an implant (e.g., knee and hip replacement, pacemaker, heart valve, or stent), a catheter, a suture, or a bone or the gut. Alternatively, or additionally, the compositions may be ingested.

The virulence suppression compositions can be administered and/or applied locally or systemically. For example, the virulence suppression compositions can be applied using a swab, cloth, sponge, nonwoven wipe, paper product such as a tissue or paper towel, or the like. When applied locally, the composition may remain where it was applied. That is, the composition may persist at the location for enough time to suppress virulence of the pathogen. In other examples, the compositions can be administered orally or intravenously. For some infections, such as those that are initiated in the gut, the compositions can be administered by drinking a solution or by swallowing a tablet or capsule.

For treatment of wounds and surgical sites, application of the virulence suppression compositions as a coating may be desirable. Alternatively, the compositions can be applied to a solid or porous support and then applied to the wound. Suitable supports include, for example, polymeric foams, polymeric films, and knitted or non-woven materials. The compositions can be used for preventing and treating both acute and chronic wound infections and can be applied to any wound surface.

The virulence suppression compositions can be administered and/or applied to reduce or prevent biofilm attachment on various surfaces. For example, the compositions can be applied to implants and catheters prior to their insertion into a mammalian body. In other examples, the compositions can be applied to bedding, surgical tables, tubing used in medical procedures, and other reusable medical equipment that contacts a mammal. In yet other examples, the compositions can be a liquid composition that is used to control or prevent biofilm populations in oral applications, such as for treating gingivitis. In still other examples, the compositions can be used to control or prevent biofilm populations in the middle ear that have been found in chronic otitis media. In yet other examples, the compositions can be used to control or prevent biofilm populations in the nose, which can result in the prevention or treatment of various infections such as those in the lungs and in blood. The compositions can often impact virulence factors either before or after biofilm formation.

In some embodiments, the virulence suppression compositions are suitable for preventing and treating urinary tract infections (e.g., administered in the form of a drink), ventilator associated pneumonia (e.g., administered in the form of a drink, tablet, or capsule), implant infections (e.g., administered by application as a coating on the implant), wounds (e.g., administered by application of a coating on the wound, whether chronic or acute), bloodstream infections (e.g., administered and/or applied to the blood-contacting tissue), mucosal tissue infections (e.g., administered in the nose), gastrointestinal tract (administered in the form of a coating, drink, tablet, or capsule), vaginal tissue (e.g., administered in the form of a coating), anastomotic tissue (e.g., administered as a coating on the surgical site to prevent anastomotic leaks), peritoneum (e.g., administered at the surgical site), sepsis, and the like. In some embodiments, where this is an existing microbial infection, the compositions may be applied over the area where the microbes are located.

In some embodiments, the virulence suppression compositions may be administered in a therapeutically effective amount. This refers to the amount of the composition that is needed to inhibit the synthesis and/or expression of one or more virulence factors by a microbe or that is enough to reduce, mitigate, or prevent a microbial infection.

In some embodiments, administering the virulence suppression compositions may suppress at least one type of virulence factor. That is, the compositions may suppress the formation or expression of various molecules that may be harmful to the mammal and/or suppresses the formation of biofilms on a foreign object such as an implant suture in the mammal. For example, the compositions can suppress the formation or expression of pyocyanin, pyoverdine, collagenase (which is often measured by breakdown of gelatin as a surrogate of collagenase activity), hemolysins, and biofilms by bacteria. The compositions may suppress, for example, the agr (accessory gene regulator) quorum sensing system in Staphylococcus aureus.

In some embodiments, the virulence suppression compositions may be supplied in the form of a kit including a container of the composition and an applicator that can be used to apply the composition to the skin. In some embodiments, the container may be a squeezable bottle or a collapsible tube, along with instructions for proper application to a treatment site or to the included applicator. In some embodiments, the kit may be supplied in sterile form in a tray, and may optionally include the application instructions along with a surgical incise drape. In some embodiments, the tray and drape may be packaged for a selected medical or surgical procedure.

In some embodiments, virulence suppression compositions can be easily manufactured and scaled up into industrial scale production. The antimicrobial composition can be formed as the ingredients are combined and mixed together, even in the absence of high shear conditions or pressure homogenization. Therefore, the composition may be prepared using any standard mixing equipment which is suitable for the preparation of liquid pharmaceutical formulations at the appropriate scale. Optionally, ultrasound treatment of the combined ingredients may be used to accelerate formation.

In some applications, the composition could include an FDA acceptable dye and in other cases, the dye could be separated from the composition within the applicator.

Objects and advantages are further illustrated by the following examples, but the particular materials and amounts thereof recited in these examples, as well as other conditions and details, should not be construed to unduly limit the present disclosure. Unless otherwise indicated, all parts and percentages are on a weight basis.

### EXAMPLES

### Materials

| Description (Abbreviation) | Source |
|---|---|
| 2X Tryptone Yeast Medium | Sigma-Aldrich Corporation, St. |
| (2X TY) | Louis, MO |
| Trypticase Soy Agar (TSA) Plates | Becton, Dickinson and Company, |
| | Franklin Lakes, NJ |
| Tryptic Soy Broth (TSB) | General Laboratory Products, |
| | Yorkville, IL |
| PRECIROL ATO 5 | Gattefosse, France |
| GELUCIRE 50/13 | Gattefosse, France |
| CAPMUL 808G | Abitec, Columbus, OH |
| GELEOL Mono and Diglycerides NF | Gattefosse, France |
| (GELEOL) | |
| pJY202 plasmid | J Bacteriol. 2004 March; 186(6): |
| | 1838-1850. |
| Ferric sulfate, zinc sulfate, | Sigma-Aldrich Corporation, |
| manganese sulfate | St. Louis, MO |
| Todd Hewitt (TH) media, | Sigma-Aldrich Corporation, |
| Dey-Engley (DE) neutralizing | St. Louis, MO |
| broth, RPMI media, erythromycin | |
| Staphylococcus aureus MN8 | BEI Resources, Manassas, VA |
| Staphylococcus aureus Xen36 | PerkinElmer, Inc., Waltham, MA |
| Pseudomonas aeruginosa MPAO1-P2 | PLoS ONE 2012: 7(8), e44326. |
| Enterococcus faecalis V583 | American Type Culture Collection, |
| (ATCC 700802) | Manassas, VA |

### Agr Reporter (Staph Aureus) Assay

Description: The expression of many virulence factors in Staphylococcus aureus is controlled by the quorum sensing accessory gene regulator (agr) system. The plasmid pJY202 is an agr reporter that contains a fusion of the agr P2-P3 region with a gene coding for GFP.

An S. aureus MN8 (pJY202) colony was picked from a TSA plate and grown overnight in TSB media with 5 g/mL erythromycin with shaking at 37° C. The culture was centrifuged at 3000×g for 5 minutes and the supernatant was removed. The bacteria were washed once with water.

Individual growth media solutions for the assay were freshly prepared by adding materials to 1X TY media with 5 μg/mL erythromycin and then adjusting the pH of each solution to about pH 6.0 (using 1M NaOH or 1M HCl). All the growth media solutions were sterile filtered when possible using a 0.2 micrometer filter.

An aliquot of each growth media solution (200 microliters) was added to the well of a 96-well black, clear-bottom plate. Samples were prepared in triplicate (n=3). The bacteria samples were resuspended in 5 mL of 1X TY media. A 3 microliter sample of resuspended S. aureus MN8 (pJY202) was added to each well. Background control wells were also prepared that did not have bacteria added to the wells. Bacterial growth was measured at 600 nm (OD600) and Agr activity was measured as fluorescent intensity at 485 nm excitation/528 nm emission. Agr activity (relative fluorescence units, RFU) was normalized to bacterial growth (OD600) for each well. The Agr activity was measured at the 24 hour time point.

### Pyoverdine Assay

Description: Pyoverdine is a fluorescent siderophore produced by Pseudomonas aeruginosa that is involved in iron scavenging and biofilm formation.

An MPAO1-P2 Pseudomonas aeruginosa colony from a TSA plate was grown overnight in TSB media (5 mL) with shaking at 37° C.

Individual growth media solutions for the assay were freshly prepared by materials to 10% TY media and then adjusting the pH of each solution to about pH 6.0 using acid or base.

Each growth media solution (200 microliters) was added to a separate well of a 96-well black, clear-bottom plate. Samples were prepared in triplicate (n=3). MPAO1-P2 bacteria were centrifuged and resuspended in 5 mL of 10% TY media and 3 microliters of resuspended bacteria was added to each well. Background control wells were also prepared that did not have bacteria added to the wells. Pyoverdine production (fluorescent intensity at 360 nm excitation/460 nm emission) and bacteria growth (OD600; i.e., absorbance at 600 nm) were measured kinetically using a plate reader (Synergy HTX Plate Reader, Biotek Instruments, Winooski, VT) with shaking at 37° C. The background values were subtracted from the respective fluorescence and absorbance measurements. The pyoverdine fluorescence values (RFU) were then normalized for bacteria growth by dividing the RFU value by the OD600 measurement. Data is shown at 24 hours.

### Time Kill Evaluation

Description: Time kill is used to assess antimicrobial activity of a test compound.

ASTM E2315-16 was used with the following specifications. Bacterial strain S. aureus Xen36 was grown overnight in Todd Hewitt (TH) media with shaking at 37° C. The next day, 0.02 mL of culture was added to 2 mL of TH media and grown for 3 hours. The bacterial culture was diluted in RPMI media to a concentration of 5×10⁸ colony forming units per mL, approximately 0.6 OD600. 0.05 mL of bacterial suspension was added to 1 mL of test formulation. At the end of each time interval, 0.1 mL was added to 12.5 mL of Dey-Engley neutralizing broth (DE broth) and vortexed to neutralize. Serial dilutions were prepared and plated onto TSA plates.

### Example 1: PEG Fatty Acid Glycerides

TABLE 1.1
Bacterial Growth And Agr Expression In Media
Containing PEG Fatty Acid Glycerides

| | S. aureus MN8 | |
|---|---|---|
| | (pJY202) (1X TY) | |
| | Growth | Agr |
| | OD600 at | Expression |
| Description | 24 hours | at 24 hours |
| TY Media control | 1.16 | 59 +/− 0.3 |
| 0.02 mM ferric sulfate, 0.02 mM manganese | 1.23 | 41 +/− 8 |
| sulfate, 0.02 mM zinc sulfate | | |
| 50 mM KH₂PO4 | 1.02 | 86 +/− 6 |
| 0.05% GELUCIRE 50/13 | 1.17 | 24 +/− 2 |
| 0.05% GELUCIRE 50/13, 25 mM KH₂PO4 | 1.07 | 13 +/− 1 |
| 0.05% GELUCIRE 50/13, 0.01 mM ferric | 1.05 | 12 +/− 1 |
| sulfate, 0.01 mM manganese sulfate, | | |
| 0.01 mM zinc sulfate | | |

TABLE 1.2
Bacterial Growth And Collagenase Production
In Media Containing PEG Fatty Acid Glycerides

| | Pseudomonas aeruginosa | |
|---|---|---|
| | P2 (10% TY) | |
| | Growth | Pyoverdine |
| | OD600 at | production at |
| Description | 24 hours | 24 hours |
| TY Media control | 0.11 | 182 +/− 61 |
| 0.05% GELUCIRE 50/13 | 0.23 | 383 +/− 82 |
| 0.05% GELUCIRE 50/13, 25 mM KH₂PO4 | 0.25 | 108 +/− 5 |
| 0.05% GELUCIRE 50/13, 0.01 mM ferric | 0.25 | 39 +/− 6 |
| sulfate, 0.01 mM manganese sulfate, | | |
| 0.01 mM zinc sulfate | | |

The results in Table 1.1 show that a PEG fatty acid glyceride (GELUCIRE 50/13) in combination with phosphate or metal salts, was able to decrease Agr expression from S. aureus compared to a no-treatment control in TY media or the single components without significantly affecting bacterial growth.

The results in Table 1.2 show that a PEG fatty acid glyceride (GELUCIRE 50/13) in combination with phosphate or metal salts, was able to decrease pyoverdine production from Pseudomonas aeruginosa P2 compared to a no-treatment control in TY media or PEG fatty acid glyceride alone without significantly affecting bacterial growth. Taken together, these results show that a PEG fatty acid glyceride plus metal salts or phosphate result in virulence suppression for two clinically important bacterial strains.

### Example 2: Fatty Acid Esters

TABLE 2.1
Bacterial Growth And Pyoverdine Production
In Media Containing Fatty Acid Esters

| | Pseudomonas aeruginosa | |
|---|---|---|
| | P2 (10% TY) | |
| | Growth | Pyoverdine |
| | OD600 at | production at |
| Description | 24 hours | 24 hours |
| TY Media control | 0.11 | 182 +/− 61 |
| 0.05% GELEOL, 25 mM KH₂PO4, | 0.22 | 230 +/− 52 |
| 0.1% TWEEN 20 | | |
| 0.05% GELEOL, 0.01 mM ferric sulfate, | 0.26 | 53 +/− 14 |
| 0.01 mM manganese sulfate, 0.01 | | |
| mM zinc sulfate, 0.1% TWEEN 20 | | |
| 0.05% GELEOL, 25 mM KH₂PO4, | 0.26 | 10 +/− 4 |
| 0.01 mM ferric sulfate, 0.01 mM | | |
| manganese sulfate, 0.01 mM zinc | | |
| sulfate, 0.1% TWEEN 20 | | |
| 0.05% PRECIROL ATO 5, 0.1% | 0.05 | 3479 +/− 1691 |
| TWEEN 20 | | |
| 0.05% PRECIROL ATO 5, 25 mM | 0.11 | 233 +/− 40 |
| KH₂PO4, 0.1% TWEEN 20 | | |
| 0.05% PRECIROL ATO 5, 0.01 mM | 0.20 | 80 +/− 9 |
| ferric sulfate, 0.01 mM manganese | | |
| sulfate, 0.01 mM zinc sulfate, 0.1% | | |
| TWEEN 20 | | |
| 0.05% PRECIROL ATO 5, 25 mM | 0.32 | 13 +/− 2 |
| KH₂PO4, 0.01 mM | | |
| ferric sulfate, 0.01 mM manganese | | |
| sulfate, 0.01 mM zinc sulfate, | | |
| 0.1% TWEEN 20 | | |

TABLE 2.2
Bacterial Growth And Agr Expression
In Media Containing Fatty Acid Esters

| | S. aureus MN8 | |
|---|---|---|
| | (pJY202) (1X TY) | |
| | Growth | Agr |
| | OD600 at | expression |
| Description | 24 hours | at 24 hours |
| TY Media control | 1.16 | 59 +/− 0.3 |
| 0.02 mM ferric sulfate, 0.02 mM manganese | 1.23 | 41 +/−8 |
| sulfate, 0.02 mM zinc sulfate | | |
| 50 mM KH₂PO4 | 1.02 | 86 +/− 6 |
| 0.05% PRECIROL ATO 5 | 1.38 | 22 +/− 4 |
| 0.05% PRECIROL ATO 5, 0.01 mM ferric | 1.10 | 12.4 +/− 0.2 |
| sulfate, 0.01 mM manganese sulfate, | | |
| 0.01 mM zinc sulfate, 0.1% TWEEN 20 | | |
| 0.05% PRECIROL ATO 5, 25 mM KH₂PO4, | 0.96 | 6.4 +/− 1.4 |
| 0.01 mM ferric sulfate, 0.01 mM manganese | | |
| sulfate, 0.01 mM zinc sulfate, 0.1% | | |
| TWEEN 20 | | |

The results in Table 2.1 show that the fatty acid esters, GELEOL or PRECIROL ATO 5, in combination with phosphate and metal salts suppressed pyoverdine production more than the fatty acid alone, fatty acid plus phosphate or fatty acid plus metal salts without decreasing bacterial growth. The results in Table 2.2 show that the fatty acid ester, PRECIROL ATO 5, in combination with phosphate and metal salts suppressed Agr expression more than the individual components or fatty acid plus metal salts without decreasing bacterial growth. Taken together, these results show that a fatty acid ester plus metal salts and phosphate result in strong virulence suppression for two clinically important bacterial strains.

### Example 3: Time Kill Experiment

TABLE 3.1
Two Minute Time Kill With S. Aureus Xen36

| | Log₁₀CFU remaining after 2 |
|---|---|
| Description | minute time kill |
| PBS control | 8.8 |
| 1.1% CAPMUL 808G | 0 |
| 0.1% CAPMUL 808G | 8.8 |

The results in Table 4.1 show that the concentration of the fatty acid containing compounds in the formulation must be carefully chosen in order to not kill bacteria in the time kill assay.

All cited references, patents, and patent applications in the above application for are herein incorporated by reference in their entirety in a consistent manner. In the event of inconsistencies or contradictions between portions of the incorporated references and this application, the information in the preceding description shall control.

1. A method of suppressing microbial virulence, the method comprising administering and/or applying a composition, the composition comprising:
a surfactant comprising a polyethylene glycol compound comprising a fatty acid moiety;
a phosphate containing compound;
a metal salt; and
an aqueous carrier
wherein the surfactant is present in the composition in an amount of between 0.01 weight percent and 1 weight percent, based on a total weight of the composition.

2. A method of suppressing microbial virulence, the method comprising administering and/or applying a composition, the composition comprising:
a surfactant comprising a polyethylene glycol compound comprising a fatty acid moiety;
at least one of a phosphate containing compound or a metal salt; and
an aqueous carrier;
wherein the surfactant is present in the composition in an amount of between 0.01 weight percent and 1 weight percent, based on a total weight of the composition.

3. A method of suppressing microbial virulence, the method comprising administering and/or applying a medical, the composition comprising:
a surfactant comprising a fatty acid containing compound;
a metal salt; and
an aqueous carrier;
wherein the surfactant is present in the composition in an amount of between 0.01 weight percent and 1 weight percent, based on a total weight of the composition.

4. A composition for suppressing microbial virulence, the composition comprising:
a surfactant comprising a polyethylene glycol compound comprising a fatty acid moiety;
a phosphate containing compound
a metal salt; and
an aqueous carrier;
wherein the surfactant is present in the composition in an amount of between 0.01 weight percent and 0.2 weight percent, based on a total weight of the composition.

5. A composition for suppressing microbial virulence, the composition comprising:
a surfactant comprising a polyethylene glycol compound comprising a fatty acid moiety;
at least one of a phosphate containing compound or a metal salt; and
an aqueous carrier;
wherein the surfactant is present in the composition in an amount of between 0.01 weight percent and 0.2 weight percent, based on a total weight of the composition.

6. The composition of claim 5, wherein the phosphate containing compound is present in the composition in an amount of between 1 mM and 50 mM, based on a total volume of the composition.

7. The composition of claim 5, wherein the metal salt is present in an amount of between 1 μM and 100 μM, based on a total volume of the composition.

8. The composition of claim 5, wherein the composition further comprises a thickening agent, and wherein the phosphate containing compound is present in an amount of between 1 mM and 1000 mM, based on a total volume of the composition.

9. The composition of claim 5, wherein the composition further comprises a thickening agent, and wherein the metal salt is present in an amount of between 1 μM and 500 μM, based on a total volume of the composition.

10. The composition of claim 5, wherein the polyethylene glycol compound comprising a fatty acid moiety is selected from the group consisting of a PEG alkyl ester, a PEG alkyl ether, a PEG alkyl amide, and a PEG 1,2 diol, and wherein an alkyl group on any PEG compound present in the composition is independently selected to have 8 to 22 carbon atoms.

11. The method of claim 3, wherein the fatty acid containing compound is selected from the group consisting of an ester, an ether, an amide, and a monoalkyl glycol, and wherein the fatty acid containing compound comprises an alkyl group that includes from 8 to 22 carbon atoms.

12. The composition of claim 5, wherein the surfactant has a hydrophilic-lipophilic balance of greater than 8 and less than 14.

13. The composition of claim 5, wherein the metal salt comprises iron.

14. The composition of claim 5, wherein the metal salt comprises a mixture of metal salts that comprises iron salts, manganese salts, and zinc salts.

15. The composition of claim 5, wherein the composition comprises part of a substance selected from the group consisting of a spray, a lotion, an ointment, a gel, a solution, an emulsion, a dispersion, a foam, a coating, a paste, a powder, a tablet, an adhesive, and a capsule.

16. The method of claim 1, wherein administering and/or applying the composition suppresses at least one virulence factor.

17. The method of claim 16, wherein the virulence factor is selected from the group consisting of Agr A expression, hemolysin, biofilm, pyoverdine, and collagenase.

18. The method of claim 1, wherein administrating and/or applying the composition comprises applying the composition to a part of a human body selected from the group consisting of skin, mucosa, tissue, a wound site, a surgical site, an implant, the gut, catheter, suture, and a bone.

19. The method of claim 1, wherein administrating and/or applying the composition reduces or inhibits virulence of at least one of gram negative Pseudomonas aeruginosa, gram positive Enterococcus faecalis, gram positive Staphylococcus aureus, or gram negative Serratia marcescens.

20. The method of claim 1, wherein the composition, when subjected to a Time-Kill test, demonstrates a log reduction of microbes of no greater than 3 logs.

21. The method of claim 1, wherein the composition does not comprise chlorhexidine salts, iodine, octenidine salts, benzalkonium chloride, parachlorometaxylenol, triclosan, hexachlorophene, fatty acid monoesters of glycerin and propylene glycol, surfactants and polymers that include a C12-C22 hydrophobe and a quaternary ammonium group, polyquaternary amines, quaternary silanes, hydrogen peroxide, or silver or silver salts or complexes.
